(19) United States
(12) Patent Application Publication
(10) Pub. No.: US 2014/0271485 A1
(43) Pub. Date: Sep. 18, 2014

(54) USE OF COELENTERAZINE FOR IMAGING INSULIN-GENERATING BETA-CELLS

(71) Applicant: The Board of Trustees of the Leland Stanford Junior University, Palo Alto, CA (US)

(72) Inventors: Laura Bronsart, Williams, AZ (US); Christopher Contag, San Jose, CA (US); Christian Stokes, Minneapolis, MN (US)

(21) Appl. No.: 14/210,551

(22) Filed: Mar. 14, 2014

Related U.S. Application Data
(60) Provisional application No. 61/787,083, filed on Mar. 15, 2013.

(51) Int. Cl. A61K 49/00

(52) U.S. Cl. CPC A61K 49/0021
USPC 424/9.6; 435/8

(57) ABSTRACT

There is a need for a broadly applicable, strain non-specific, bioluminescent imaging tool that will enable researchers to study a β-cell mass in the context of development, disease or transplantation. The disclosure, therefore, encompasses embodiments of a method of identifying a non-diabetic pancreatic β-cell, the method comprising the steps of: delivering to a pancreatic β-cell a composition comprising a coelenterazine; allowing the β-cell to generate a coelenterazine-dependent bioluminescent signal; and identifying the cell as a non-diabetic pancreatic β-cell by detecting the emitted coelenterazine-dependent bioluminescent signal.

### CROSS-REFERENCE TO RELATED APPLICATION

This application claims priority to U.S. Provisional Application No. 61/787,083, entitled “APPLICATION OF THE LUCIFERASE SUBSTRATE COELENTERAZINE FOR IMAGING BETA-CELL MASS” filed on Mar. 15, 2013, the entirety of which is herein incorporated by reference in its entirety.

### TECHNICAL FIELD

The present disclosure is generally related to a method of distinguishing pancreatic β-cells having the ability to generate insulin from non-insulin producing cells.

### BACKGROUND

A continuing limitation to the advancement of type I and II diabetes research is the inability to longitudinally quantify the insulin-producing cells (or β-cells) of the pancreas. The standard method is bioluminescence imaging using β-cell firefly luciferase expression and the substrate luciferin. Although this technique does quantify β-cell masses, it cannot be efficiently applied to a variety of transgenic mouse strains. Thus, there is a need for a broadly applicable, strain non-specific, bioluminescent imaging tool that will enable researchers to study a β-cell mass in the context of development, disease or transplantation.

An important area in diabetes research is the development of a targeted imaging probe that is highly sensitive, specific and suitable for diagnostic β-cell mass imaging. In the process of evaluating a diabody-based, β-cell imaging tool, a novel application for the luciferase substrate coelenterazine was found, namely the ability to detect a functional β-cell mass.

### SUMMARY

One aspect of the disclosure, therefore, encompasses embodiments of a method of identifying a non-diabetic pancreatic β-cell, the method comprising the steps of: delivering to a pancreatic β-cell a composition comprising a coelenterazine; allowing the β-cell to generate a coelenterazine-dependent bioluminescent signal; and identifying the cell as a non-diabetic pancreatic β-cell by detecting the emitted coelenterazine-dependent bioluminescent signal.

In embodiments of this aspect of the disclosure, the method can further comprise the steps of: delivering the coelenterazine to the pancreas of a mammal; and identifying the mammal as having non-diabetic β-cells by detecting the emission of a coelenterazine-dependent bioluminescent signal by the pancreas of the mammal.

In embodiments of this aspect of the disclosure, the method can further comprise the steps of: determining the location of the non-diabetic pancreatic β-cells within the mammal by generating an image of the emitted coelenterazine-dependent bioluminescent signal and locating the signal relative to the mammal.

Another aspect of the disclosure encompasses embodiments of a method of imaging non-diabetic pancreatic β-cells in the pancreas of a mammalian subject, comprising: (i) administering a pharmaceutically acceptable composition comprising a coelenterazine and a pharmaceutically acceptable carrier to a first mammal; (ii) allowing the coelenterazine to be delivered to the pancreas of the first mammal; (iii) generating an image of a coelenterazine-dependent bioluminescent signal by the first mammal; (iv) repeating the steps (i)-(iii) on a second mammal, wherein the second mammalian subject is a non-diabetic animal; and (v) determining that the first mammal has a reduced population of β-cells and a substantial likelihood of being diabetic by determining the relative intensities of the images derived from the first and second mammals, whereby a reduction in the intensity of the detectable bioluminescent signal from the first mammal compared to the intensity of the signal from the second mammal indicates that the first mammal has a reduced population of β-cells and has a substantial likelihood of being diabetic.

### BRIEF DESCRIPTION OF THE DRAWINGS

Aspects of the present disclosure will be more readily appreciated upon review of the detailed description of its various embodiments, described below, when taken in conjunction with the accompanying drawings. The drawings are described in greater detail in the description and examples below.

FIG. 1A is a digital image illustrating a cell plate assay. 10⁵ cells of each cell line were washed two times in serum-free RPMI and plated in a 96-well black plate. Coelenterazine was added to a final concentration of 10 μM and the plate imaged. RIN5F is a β-cell line and the remainder are control cell lines.

FIG. 1B is a graph illustrating the quantification of the bioluminescence signals of FIG. 1A. Error bars represent the standard error.

FIG. 2A is a digital image illustrating bioluminescence images of excised tissues from 3 mice in 1 mL PBS with 20 μg coelenterazine and imaged immediately.

FIG. 2B is a graph illustrating the quantification of the bioluminescence signals shown in FIG. 2A. Significantly greater signals were detected from the pancreas compared to all other tissues. Error bars represent the standard error and significance is defined as P<0.05.

FIG. 3A is a digital image, for the in vivo characterization of a coelenterazine (CTZ) pancreatic signal, showing the bioluminescence imaging of mice that were fasted for 6 h or received 2 g/kg intraperitoneal glucose and maintained on 10% sucrose water for 4 h. Mice then received 40 μg CTZ intravenously and were imaged immediately.

FIG. 3B is a graph illustrating the quantification of the bioluminescence signal detected in FIG. 3A. Error bars represent the standard error.

FIG. 3C is a digital image, for the in vivo characterization of a coelenterazine (CTZ) pancreatic signal, showing the bioluminescence imaging of mice treated with glucose followed by administration of 0.56, 1.4, or 3.5 mg/kg CTZ intravenously.

FIG. 3D is a graph illustrating the quantification of the signal detected in FIG. 3C at 1 min intervals following CTZ injection.

FIGS. 4A-4D illustrate the results from a streptozotocin-induced diabetic mouse model. Mice were treated with 200 mg/kg of streptozotocin (STZ) or PBS on day 0.

FIG. 4A shows digital bioluminescence imaging of live mice. Top Panel: 3-days post-treatment, mice were administered 2 g/kg glucose and maintained on 10% sucrose water. Four h later, mice were intravenously injected with 1.4 mg/kg of coelenterazine and imaged. Bottom panel: Bioluminescence imaging of the excised pancreases collected from the mice depicted in top panel.

FIG. 4B is a graph illustrating the difference (P=0.0265) observed in blood glucose values measured on day 3.

FIG. 4C is a graph illustrating the quantification of the in vivo bioluminescence signal of the pancreases and of the backgrounds from mice depicted in FIG. 4A. A significant difference (P=0.0000) between the two groups was observed for pancreatic signal but not for background signal, indicating reduction in signal intensity was pancreas specific.

FIG. 4D is a graph illustrating the measured blood glucose and observed pancreatic signal. The correlation was significant (P=0.0317) and depicts the previously described severe beta cell loss that occurs prior to hyperglycemia.

FIG. 5A is a digital image illustrating the in vivo characterization of the coelenterazine (CTZ) pancreatic signal imaging kinetics, showing the bioluminescence of a mouse that was sequentially imaged following intravenous administration of 10 mg/kg native coelenterazine.

FIG. 5B is a graph illustrating the quantification of the bioluminescence signal detected in FIG. 5A. “Body” relates to the signal measured within a region of interest that incorporated the entire mouse. “Lungs” relates to the signal within a region of interest that incorporated the thoracic signal. “Pancreas” relates to the signal within a region of interest that incorporated the abdominal signal.

FIG. 6A is a digital image illustrating the in vivo characterization of the coelenterazine (CTZ) pancreatic signal detected using various derivatives of coelenterazine, showing the bioluminescence of a representative mouse from each group. Mice received 2 g/kg intraperitoneal glucose and were maintained on 10% sucrose water for 4 h. Mice then received 25 μg CTZ intravenously and were imaged immediately.

FIG. 6B is a graph illustrating the quantification of the bioluminescence signal detected in FIG. 6A. Error bars represent the standard error.

### DESCRIPTION OF THE DISCLOSURE

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law, allowing for the presence of more than that which is recited so long as basic or novel characteristics of that which is recited is not changed by the presence of more than that which is recited, but excludes prior art embodiments.

### DEFINITIONS

In describing and claiming the disclosed subject matter, the following terminology will be used in accordance with the definitions set forth below.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

The terms “delivering” and “delivering to a cell” as used herein refer to the direct targeting of a cell with a small molecule compound or by incubation of the cell or cells with said effector ex vivo or in vitro. The terms further refer to the act of contacting a cell or a population of cells with a small molecule according to the disclosure. The terms also refer to administering the effector, such as, but not limited to, coelenterazine to an animal for systemic or localized distribution of the effector within a whole animal.

The term “population” of cells refers to a plurality of cells obtained by a particular isolation of the starting cells or culture procedure. Properties of a cell population are generally defined by a percentage of individual cells having the particular property (e.g. the percentage of cells staining positive for a particular marker) or the bulk average value of the property when measured over the entire population (e.g. the amount of mRNA in a lysate made from a cell population, or percentage of cells positive for a histochemical marker, such as Ngn3, Pax6, Insulin or Glucagon).

The term “subject” as used herein refers to an animal, preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In aspects of the disclosure, the term includes domestic animals bred for food or as pets, including equines, bovines, sheep, poultry, fish, porcines, canines, felines, and zoo animals, goats, apes (e.g. gorilla or chimpanzee), and rodents such as rats and mice.

The term “β-cell (beta cell)” is a type of cell in the pancreas and is located in the Islets of Langerhans, its main function being to synthesize and secrete the hormone insulin that controls the quantities of glucose circulating in blood. The cells normally produce the hormone that is released daily at about 40 to 50 Units. The Islets have several hundred of these cells. Insulin is stored and available in vesicles within the cytoplasm of these cells, and is secreted when needed. If some β-cells are affected, that is, if only between 10% and 20% remain in good condition, the symptoms of diabetes will be displayed.

“Type II diabetes (T2D)” means a condition characterized by insulin resistance which may be combined with relatively reduced insulin secretion. “Type 1 diabetes (T1D)” means a condition characterized by loss of the insulin-producing β-cells of the Islets of Langerhans in the pancreas leading to insulin deficiency. This type of diabetes can be further classified as immune-mediated or idiopathic. Type 1 diabetes can affect children or adults but was traditionally termed “juvenile diabetes” because it represents a majority of the diabetes cases in children.

TD2 occurs with more frequency than TD1, and happens in people after 40 years, although pre-teens with obesity may encounter TD2. In this type of diabetes, the ability to produce insulin doesn't disappear but the cells of the body offer resistance to the action of the hormone.

The term “coelenterazine” as used herein refers to the compound 6-(4-hydroxyphenyl)-2-[(4-hydroxyphenyl)methyl]-8-(phenylmethyl)-7H-imidazo[3,2-a]pyrazin-3-one, which is natural coelenterazine. The term may further refer to derivatives of coelenterazine, including, but not limited to, modified colenterazines, available from PROMEGA CORPORATION®, Madison, Wis. and from MOLEUCLAR PROBES, INC., Eugene, Oreg. Coelenterazines may also be synthesized as described, for example, in Shimomura et al., (1989) Biochem. J. 261: 913-20, 1989; Inouye et al., (1997) Biochem. Biophys. Res. Comm. 233: 349-53; and Teranishi et al., (1997) Anal. Biochem. 249: 37-43.

Both natural coelenterazine and modified coelenterazines can function as luminophores with luminogenic proteins. Factors which affect this interaction include the identity of the luminogenic protein and the characteristics of the surrounding environment. For example, the interaction of a luminogenic protein, such as Renilla or Oplophorus luciferase or aequorin, with a coelenterazine, in the presence of O₂ and at least a trace of cofactor Ca²⁺, will produce luminescence. The coelenterazine is oxidized to its corresponding coelenteramide during this process.

The term “coelenterazine-dependent bioluminescent signal” as used herein refers to a bioluminescent signal having a wavelength in the visible spectrum and generated by contact of a coelenterazine with a non-diabetic β-cell. Within the context of the methods of the disclosure, the signal may be generated by contacting an isolated β-cell, a population of isolated primary β-cells, a population of cultured β-cells, an isolated organ, most typically a pancreas but also including an organic comprising homo- or xenografted β-cells, or such an organ within an animal, with a composition that comprises a coelenterazine in an effective amount that will lead to the generation of a detectable bioluminescent signal by the β-cell or cells.

The term “bioluminescence” as used herein refers to a type of chemiluminescent, emission of light by biological molecules, particularly proteins. The essential condition for bioluminescence is molecular oxygen, and typically an enzyme that, in the presence of molecular oxygen, transforms a substrate such as coelenterazine to an excited state, which upon return to a lower energy level releases the energy in the form of light.

The term “mammal” as used herein refers to any member of the phylogenetic group, including humans that may generate a detectable bioluminescence from non-diabetic beta-cells in contact with a coelenterazine.

The term “pharmaceutically-acceptable carrier” as used herein refers to a pharmaceutically-acceptable material, composition or vehicle, such as a liquid or solid filler, diluent, excipient, solvent or an encapsulating material such as liposomes, polyethylene glycol (PEG), PEGylated liposomes, nanoparticles and the like, involved in carrying or transporting the subject compositions or therapeutic agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### Description

Type I and type II diabetes are chronic, debilitating and costly diseases that are increasing in prevalence worldwide. A significant limitation to the early diagnosis of type I (insulin-dependent) diabetes and staging of type II (insulin-independent) diabetes is an inability to quantify the β-cell mass of the pancreas. These β-cells, located in the pancreas, are responsible for secreting the hormone insulin in response to an elevation in blood glucose levels. Type I diabetes is associated with immune destruction of β-cells while late-stage type II diabetes is associated with progressive loss of β-cell mass subsequent to insulin resistance. The major diagnostic criterium for these diseases is hyperglycemia; however, hyperglycemia is not evident until about 90% of the functional β-cell mass is loss. Thus, there is a significant clinical need for β-cell mass imaging agents that would allow an assessment of the level of existing functional (i.e. non-diabetic) β-cells in the pancreas and, therefore, allow the selection of suitable treatment or prophylactic protocols to reduce or prevent the onset of diabetes.

Similarly, the detection of a β-cell mass in research models of type I and type II diabetes is currently limited. One of the most commonly applied techniques is post-mortem histology. This necessarily limits the assessment to a single time point, which significantly increases the number of animals needed for a given study and fails to utilize the power of longitudinal studies.

A transgenic mouse model that does permit longitudinal analysis of the β-cell mass uses the β-cell-specific expression of the firefly luciferase enzyme. Upon administration of the enzyme substrate luciferin, β-cell bioluminescence can be detected by a super-cooled CCD camera and quantified. Though this technique does permit quantification of the β-cell mass, it has no capacity for translation to human medicine. This is because it depends on the prior generation of the luciferase-expressing transgene animal, which is not possible with humans. Additionally, due to the transgenic nature of the model, its use is limited to only those mouse lines or, only after prolonged cross breeding, to another transgenic mouse line of interest.

The disclosure, however, encompasses a novel method for imaging the β-cell mass in animals and humans. Coelenterazine is a small molecule substrate typically used in conjunction with several bioluminescent luciferase enzymes including Gaussia and Renilla luciferases. This disclosure describes the use of coelenterazine or coelenterazine derivatives as a single agent (without requiring the addition of a luciferase enzyme) to image beta cell mass. It has now been found that coelenterazine administered to mice allows the generation of a pancreatic-specific bioluminescent signal that may be usefully quantified, or used as both an indicator of the presence of non-diabetic β-cells and as a means to provide images of the location of such cells in an animal body. This signal is reduced in type I diabetic mice, which have a reduction in β-cell mass, indicating that coelenterazine can be used to image β-cell mass in vivo.

Accordingly, this disclosure encompasses the use of coelenterazine and coelenterazine-like small molecules for imaging a β-cell mass in mammals. It is further contemplated to be within the scope of the disclosure for the coelenterazine or its derivatives to be have additional labeling agents attached to its structure and for small molecules developed using a coelenterazine-like small molecule structure to generate its β-cell specificity.

The disclosure describes the use of coelenterazine or coelenterazine derivatives, therefore, for the quantification of β-cell mass in human patients and/or subjects and research models. The disclosure further encompasses the use of coelenterazine, or any derivative thereof, for imaging βcells anywhere in the body or ex-vivo. Although β-cells are normally found in the pancreas, in cases of transplantation in humans they are usefully localized to the liver and in mice to the kidney capsule (also, sometimes subcutaneously or orbitally). Thus, it would be advantageous to image β-cell mass independent of their location. It is further contemplated that coelenterazine may be linked to labels such as a radionuclide for detection of the β-cells by other than just bioluminescence labeling and which could require such as PET or SPECT detection that may be used for imaging in humans.

In vitro experiments have revealed that a variety of rat insulinoma cell lines could generate a greater coelenterazine-dependent bioluminescence signal relative to other cell lines and in the absence of any additional reagents. Additionally, upon intravenous injection into mice, a mid-upper abdominal bioluminescence signal was detected, and ex vivo imaging revealed a significantly elevated pancreatic signal compared to other examined non-insulin-producing tissues of the same animal. This detectable signal intensity proved to be coelenterazine dose-dependent.

To further study the in vivo characteristics of the observed signal, the dependency upon β-cell functioning was tested. It was determined that insulin-secreting β-cells produced a greater signal compared to those cells that were quiescent due to fasting. These results supported the conclusion that the in vivo coelenterazine signal wasp-cell specific.

To conclusively determine the β-cell specificity of the signal, coelenterazine was used to image the pancreas of mice that received the β-cell toxic agent streptozotocin (STZ) versus a Phosphate-Buffered Saline (PBS) control in which the β-cells were functional. There was a significant reduction in pancreatic signal in STZ-treated mice with both in vivo and ex vivo imaging of the pancreas. Furthermore, there was a significant and inverse correlation between the pancreatic signal and the corresponding blood glucose levels with findings similar to previous observations made in type II diabetic humans whose pancreatic mass was determined at autopsy; there appears to be a severe reduction in the β-cell mass prior to the development of hyperglycemia. These results demonstrated a novel use of coelenterazine to detect the functional β-cell mass in the STZ, type-1-diabetic mouse model.

One aspect of the disclosure, therefore, encompasses embodiments of a method of identifying a non-diabetic pancreatic β-cell, the method comprising the steps of: delivering to a pancreatic β-cell a composition comprising a coelenterazine; allowing the β-cell to generate a coelenterazine-dependent bioluminescent signal; and identifying the cell as a non-diabetic pancreatic β-cell by detecting the emitted coelenterazine-dependent bioluminescent signal.

In embodiments of this aspect of the disclosure, the method can further comprise the steps of: delivering the coelenterazine to the pancreas of a mammal; and identifying the mammal as having non-diabetic β-cells by detecting the emission of a coelenterazine-dependent bioluminescent signal by the pancreas of the mammal.

In embodiments of this aspect of the disclosure, the method can further comprise the steps of: determining the location of the non-diabetic pancreatic β-cells within the mammal by generating an image of the emitted coelenterazine-dependent bioluminescent signal and locating the signal relative to the mammal.

Another aspect of the disclosure encompasses embodiments of a method of imaging non-diabetic pancreatic β-cells in the pancreas of a mammalian subject, comprising: (i) administering a pharmaceutically acceptable composition comprising a coelenterazine and a pharmaceutically acceptable carrier to a first mammal; (ii) allowing the coelenterazine to be delivered to the pancreas of the first mammal; (iii) generating an image of a coelenterazine-dependent bioluminescent signal by the first mammal; (iv) repeating the steps (i)-(iii) on a second mammal, wherein the second mammalian subject is a non-diabetic animal; and (v) determining that the first mammal has a reduced population of β-cells and a substantial likelihood of being diabetic by determining the relative intensities of the images derived from the first and second mammals, whereby a reduction in the intensity of the detectable bioluminescent signal from the first mammal compared to the intensity of the signal from the second mammal indicates that the first mammal has a reduced population of β-cells and has a substantial likelihood of being diabetic.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLES

### Example 1

In Vitro Experiments for the Imaging Cell Lines:

In vitro experiments for the imaging cell lines may be completed on the day of cell collection or following incubation. If imaging is performed following prolonged incubation combined with comparisons between dissimilar cell lines, it is important to consider the likelihood of there being a different division rate for each cell line.

For same-day imaging, the cells are collected by a method dictated by the particular cell line. Cells are washed twice in imaging buffer by repeated resuspension and centrifugation (a variety of buffers have proven effective, including: PBS, DPBS and Krebs). It is necessary that the chosen buffer does not contain proteins including albumin. The desired concentration of cells is added to an imaging plate.

Coelenterazine is resuspended to a concentration of about 1-10 mg/mL in 100% ethanol and then diluted in imaging buffer to achieve the desired imaging concentration. A final imaging concentration of 10 μM is suitable for most in vitro experiments.

The coelenterazine is added to wells to be imaged and the signal detected using a bioluminescence detection system such as an IVIS. The signal typically reaches a stable maximum approximately 15-45 min following coelenterazine addition. For imaging following incubation and cell adherence to imaging wells, wells are carefully washed twice in imaging buffer by repeated aspiration and reapplication and imaged as described.

### Example 2

In vivo imaging of beta cell masses in mice requires selection of the time of imaging since some circadian fluctuation in the signal intensity is found. A standardized anesthetic protocol with a defined anesthetic dose and induction period (if using inhalant anesthetics) is also required.

Coelenterazine is prepared at a concentration of 10 mg/mL in 100% ethanol. The coelenterazine may be further diluted in a pharmaceutically acceptable carrier selected as suitable for injection into an animal subject for imaging. For example, but not intended to be limiting, an injection buffer of a 5% glucose solution has proven effective. It is important to protect the imaging solution from light and that is prepared immediately prior to use.

Following anesthetic induction, the imaging solution is intravenously administered at a dose of about 1-10 mg/kg and imaging is begun immediately in using an in vivo bioluminescence imaging system. The pancreatic signal intensity is maximum 2-5 minutes post-injection.

We claim:

1. A method of identifying a non-diabetic pancreatic β-cell, the method comprising the steps of:
delivering to a pancreatic β-cell a composition comprising a coelenterazine;
allowing the β-cell to generate a coelenterazine-dependent bioluminescent signal; and
identifying the cell as a non-diabetic pancreatic β-cell by detecting the emitted coelenterazine-dependent bioluminescent signal.

2. The method of claim 1, wherein the method further comprises the steps of:
delivering the coelenterazine to the pancreas of a mammal; and
identifying the mammal as having non-diabetic β-cells by detecting the emission of a coelenterazine-dependent bioluminescent signal by the pancreas of the mammal.

3. The method of claim 2, further comprising the steps of:
determining the location of the non-diabetic pancreatic β-cells within the mammal by generating an image of the emitted coelenterazine-dependent bioluminescent signal and locating the signal relative to the mammal.

4. A method of imaging non-diabetic pancreatic β-cells in the pancreas of a mammalian subject, comprising:
(i) administering a pharmaceutically acceptable composition comprising coelenterazine and a pharmaceutically acceptable carrier to a first mammal;
(ii) allowing the coelenterazine to be delivered to the pancreas of the first mammal;
(iii) generating an image of a coelenterazine-dependent bioluminescent signal by the first mammal;
(iv) repeating the steps (i)-(iii) on a second mammal, wherein the second mammalian subject is a non-diabetic animal; and
(v) determining that the first mammal has a reduced population of β-cells and a substantial likelihood of being diabetic by determining the relative intensities of the images derived from the first and second mammals, whereby a reduction in the intensity of the detectable bioluminescent signal from the first mammal compared to the intensity of the signal from the second mammal indicates that the first mammal has a reduced population of β-cells and has a substantial likelihood of being diabetic.
